(19) United States
(12) Patent Application Publication
(10) Pub. No.: US 2025/0100954 A1
(43) Pub. Date: Mar. 27, 2025

(54) SYSTEMS AND METHODS FOR CARRYING OUT A METATHESIS REACTION

(71) Applicant: SABIC GLOBAL TECHNOLOGIES B.V., BERGEN OP ZOOM (NL)

(72) Inventors: Saud ALKHUDEER, Bangalore (IN); Aspi Kersasp KOLAH, Bangalore (IN); Vidya Sagar GUGGILLA, Bangalore (IN); Venugopal BV, Bangalore (IN); Rajeshwer DONGARA, Bangalore (IN)

(73) Assignee: SABIC GLOBAL TECHNOLOGIES B.V., BERGEN OP ZOOM (NL)

(21) Appl. No.: 18/725,121

(22) PCT Filed: Dec. 23, 2022
(86) PCT No.: PCT/EP2022/087742

§ 371 Date: Jun. 27, 2024

(30) Foreign Application Priority Data
Dec. 31, 2021 (IN) 202141062110

(51) Int. Cl. C07C 5/25; C07C 7/04

(52) U.S. Cl. CPC C07C 5/2512; C07C 7/04; C07C 2521/04

(57) ABSTRACT

Systems and methods for carrying out metathesis reaction by utilizing one or more side reactors to a reactive distillation column is disclosed. The one or more side reactors is used to effect a reactive pump-around process. The systems and methods are used for equilibrium limited reactions such as the metathesis of C₄ olefins.

### CROSS REFERENCE TO RELATED APPLICATIONS

None.

### FIELD OF INVENTION

The present disclosure generally relates to carrying out a metathesis chemical reaction in a production process. More specifically, the present disclosure relates to the production of olefins by metathesis reaction by utilizing one or more side reactors to a reactive distillation column.

### BACKGROUND OF THE INVENTION

Olefins are very important hydrocarbon products-they are the building block for a variety of other petrochemicals such as plastics, resins, fibers and solvents. Consequently, several petrochemical plants worldwide are designed to produce olefins by various different processes. One such process—olefin metathesis—is the simultaneous reaction of olefin carbon-carbon double bond cleavage with rearrangement to form two new olefins, typically having a lower molecular weight and a higher molecular weight.

Metathesis reactions are in general equilibrium limited chemical reactions, meaning that the reaction cannot achieve complete conversion in a single stage reactor like a batch reactor, a continuous stirred-tank reactor (CSTR), or a packed bed continuous plug flow reactor. Reactive distillation (RD) is a proven process intensification method which can save downstream separation costs by performing separation and reaction simultaneously in a single integrated unit operation. Reactive distillation enhances reaction yields by constantly removing one or more of the products from the reaction mixture and creating favorable zones for the equilibrium limited chemical reaction. Metathesis reactions are ideally suited for reactive distillation applications since reaction conditions are ambient to moderate temperature and the reactions take place typically in the liquid phase. Usually, the boiling point of the products from metathesis reactions straddle the boiling point of the reactants and reactive azeotropes are avoided thereby facilitating easy multicomponent distillation of the reactants and products.

Reactive distillation is a versatile system used commercially for driving equilibrium limited chemical reactions to completion. For example, reactive distillation has been applied to systems such as etherification for synthesis of MTBE, ETBE, TAME, and ETEE and esterification systems for synthesis of methyl acetate and ethyl acetate. However the potential benefits of applying reactive distillation for equilibrium limited chemical reactions is taxed by extreme complexities in the process development and design stage.

Notwithstanding the limitations of reactive distillation, metathesis is a preferred route for valorization of low value olefins like C₄ olefins into high value olefins that have a market demand, such as propylene and 1-hexene. In recent years, on demand synthesis of propylene and 1-hexene has been garnering great attention.

### BRIEF SUMMARY OF THE INVENTION

The inventors have discovered an intensification scheme for metathesis, e.g., C₄ metathesis, using an integrated reactive distillation column with reactive pump-arounds for the metathesis. In this way, the present disclosure provides a versatile route for on demand synthesis of olefins such a propylene and 1-hexene. An external side reactor, which, in embodiments of the disclosure, comprises a plug flow reactor, is used for the purpose of the reactive pump-around.

Embodiments of the disclosure include a method of producing a product by metathesis reaction. The method includes distilling a distillation column feed stream in a distillation column, withdrawing a side stream from the distillation column and reacting, in a first side reactor, reactants of the side stream, by metathesis reaction, to form a first side reactor effluent stream. The method also includes distilling the first side reactor effluent stream in the distillation column and flowing, from the distillation column, a stream comprising product.

Embodiments of the disclosure include a method of producing a product by metathesis reaction. The method includes contacting, in a pre-distillation reactor, reactants of a feed stream in the presence of a catalyst and thereby carrying out a metathesis reaction that forms a distillation column feed stream comprising product and unreacted reactants. The method also includes distilling the distillation column feed stream in a distillation column, withdrawing a side stream from the distillation column, and contacting, in a first side reactor, unreacted reactants of the side stream, by metathesis reaction, to form a first side reactor effluent stream. The method also includes distilling the first side reactor effluent stream in the distillation column and flowing, from the distillation column, a stream comprising product.

Embodiments of the disclosure include a method of producing ethylene and/or propylene by metathesis reaction of C₄ hydrocarbons. The method includes contacting, in a pre-distillation reactor, reactants of a feed stream comprising C₄ hydrocarbons in the presence of a catalyst comprising Re/γ-Alumina and/or K/γ-Alumina and thereby carrying out a metathesis reaction that forms a distillation column feed stream comprising ethylene and/or propylene and unreacted C₄ hydrocarbons. The method also includes distilling the distillation column feed stream in a distillation column, withdrawing a side stream from the distillation column and contacting, in a first side reactor, unreacted C₄ hydrocarbons of the side stream in the presence of a catalyst comprising Re/γ-Alumina and/or K/γ-Alumina and thereby carrying out a metathesis reaction, to form a first side reactor effluent stream. The method further includes distilling the first side reactor effluent stream in the distillation column and flowing, from the distillation column, a stream comprising ethylene and/or propylene.

The following includes definitions of various terms and phrases used throughout this specification.

The terms “about” or “approximately” are defined as being close to as understood by one of ordinary skill in the art. In one non-limiting embodiment the terms are defined to be within 10%, preferably, within 5%, more preferably, within 1%, and most preferably, within 0.5%.

For the purposes of this disclosure, “X, Y, and/or Z” can be construed as X only, Y only, Z only, or any combination of two or more items X, Y, and Z (e.g., XYZ, XY, XZ, YZ).

The terms “wt. %”, “vol. %” or “mol. %” refer to a weight, volume, or molar percentage of a component, respectively, based on the total weight, the total volume, or the total moles of material that includes the component. In a non-limiting example, 10 moles of component in 100 moles of the material is 10 mol. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification, include any measurable decrease or complete inhibition to achieve a desired result.

The term “effective,” as that term is used in the specification and/or claims, means adequate to accomplish a desired, expected, or intended result.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The process of the present disclosure can “comprise,” “consist essentially of,” or “consist of” particular ingredients, components, compositions, etc., disclosed throughout the specification.

The term “primarily,” as that term is used in the specification and/or claims, means greater than any of 50 wt. %, 50 mol. %, and 50 vol. %. For example, “primarily” may include 50.1 wt. % to 100 wt. % and all values and ranges there between, 50.1 mol. % to 100 mol. % and all values and ranges there between, or 50.1 vol. % to 100 vol. % and all values and ranges there between.

Other objects, features and advantages of the present disclosure will become apparent from the following figures, detailed description, and examples. It should be understood, however, that the figures, detailed description, and examples, while indicating specific embodiments of the disclosure, are given by way of illustration only and are not meant to be limiting. Additionally, it is contemplated that changes and modifications within the spirit and scope of the disclosure will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

For a more complete understanding, reference is now made to the following descriptions taken in conjunction with the accompanying drawings, in which:

FIG. 1 shows a system for producing a product by metathesis reaction, according to embodiments of the disclosure;

FIG. 2 shows a method for producing a product by metathesis reaction, according to embodiments of the disclosure;

FIG. 3 shows a system for producing a product by metathesis reaction according to embodiments of the disclosure;

FIG. 4 shows a catalyst loading diagram for experiments for the metathesis reaction on a C₄ feed and a plot of temperature and pressure conditions versus time;

FIGS. 5A to 5J show results of an experiment involving the metathesis reaction on a C₄ feed;

FIG. 6 shows a plug flow reactor profile used in experiments;

FIG. 7A to 7J show the experimental outlet reactor composition vs the Aspen predicted values;

FIG. 8 shows the setup of the Aspen Plus program for evaluating the performance of commercial plant process using a standalone plug flow reactor and a reactive pump-around process;

FIG. 9 shows the Aspen Plus simulations for both a stand-alone plug flow reactor and a reactive pump-around process using two external side reactors; and

FIG. 10 shows the Aspen Plus simulations for both a stand-alone plug flow reactor and a reactive pump-around process using two external side reactors.

### DETAILED DESCRIPTION OF THE INVENTION

Embodiments of the present disclosure involve a process intensification scheme for metathesis reactions using a distillation column with one or more external side reactors as a reactive pump-around process. The external side reactor(s) that is used for the purpose of the reactive pump-around can be a plug flow reactor, according to embodiments of the disclosure. In embodiments of the disclosure, reactive pump-arounds using external side-reactors can be useful in (1) carrying out reactions for systems which are equilibrium limited, (2) removal of exothermic heat of reactions by utilizing such heat for vaporizing of one or more of the components of the reaction mixture, and (3) maximizing intermediate reaction products for sequential reactions. According to embodiments of the disclosure, the catalyst of the external side reactor can be regenerated at will and at desired high temperatures when the catalyst deactivates. At the liquid side draw withdrawal, in embodiments of the disclosure, the process can involve the use of two external side reactors, one being in operation while the other is being regenerated. This novel yet versatile technology is applicable for chemical reactions that are equilibrium limited and is broadly applicable to metathesis reactions of C₂ to C₁₂ olefins, isomerization reactions of C₆ compounds, etherification reactions of C₄ and C₅ olefins with alcohols like methanol, ethanol, isoamylalcohol etc. to form gasoline additives which increase octane number of the fuel pool, esterification, acetalization, isomerization reactions etc. In this disclosure, a process for metathesis of olefins such as C₄ olefins is described. The C₄ olefins can be 1-butene or trans/cis-2-butene. The metathesis reactions can be either of self or cross type. In self-type metathesis, two molecules of one reactant are converted and two products are formed, in cross type metathesis two different products are reacted and converted into two products. The general scheme for self and cross metathesis reactions are shown below.

$$Self metathesis 2 A ↔ C + D where the relative volatilities are {α}_{C} > {α}_{A} > {α}_{D}$$

$$Cross metathesis A + B ↔ C + D$$

The reaction mechanism for the specific process described in this disclosure are shown below.

Isomerization: (t/c)2−butene⇔1−butene

Cross-metathesis: (t/c)2−butene+1−butene⇔propylene+(t/c)2−pentene

1−butene+propylene⇔ethylene+(t/c)2−pentene

1−butene+(t/c)2−pentene⇔propylene+(t/c)3−hexene

Self-metathesis:2*1−butene⇔ethylene+(t/c)3−hexene

### Systems for Producing a Product by Metathesis Reaction

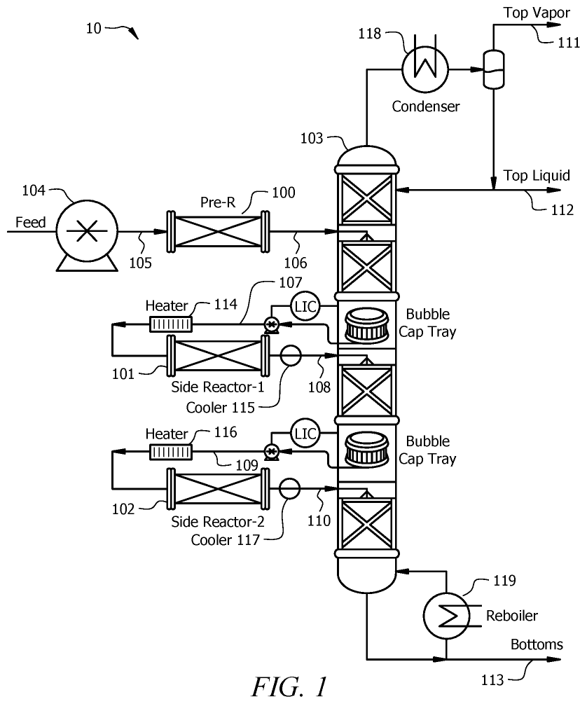

FIG. 1 shows system 10 for producing a product by metathesis reaction, according to embodiments of the disclosure. According to embodiments of the disclosure, system 10 includes reactive distillation column 103, which includes therein a catalyst and is configured to react reactants and concurrently separate and remove one or more of the products from the reaction mixture. In this way, reactive distillation column 103 can create favorable zones for equilibrium limited reactions such as metathesis reactions. In embodiments of the disclosure, reactive distillation column 103 can have the catalyst in a packed configuration and/or a tray configuration in the different distillation column stages. Catalyst loading configurations, according to embodiments of the disclosure, include spherical basket, cylindrical containers for catalyst particles, wire gauze envelopes, horizontally disposed gutters, and horizontally disposed wire gauzed tubes.

As shown in FIG. 1, in embodiments of the disclosure, system 10 includes side reactor 101 and side reactor 102. According to embodiments of the disclosure, side reactor 101 and side reactor 102 are configured to react side stream 107 and side stream 109, respectively, to produce side reactor effluent stream 108 and side reactor effluent stream 110, respectively, as shown in FIG. 1.

Side reactor 101 and side reactor 102 may each include catalyst therein (e.g., Re/γ-Alumina and/or K/γ-Alumina) for catalyzing a metathesis reaction. According to embodiments of the disclosure, in system 10, the same type of metathesis reaction occurs in reactive distillation column 103, side reactor 101, and/or side reactor 102. An inlet of side reactor 101 is in fluid communication with an outlet of reactive distillation column 103 such that side stream 107 can flow from reactive distillation column 103 to side reactor 101 and an outlet of side reactor 101 is in fluid communication with an inlet of reactive distillation column 103 such that side reactor effluent stream 108 can flow from side reactor 101 to reactive distillation column 103, as shown in FIG. 1. Similarly, side reactor 102 is in fluid communication with an outlet of reactive distillation column 103 such that side stream 109 can flow from reactive distillation column 103 to side reactor 102 and an outlet of side reactor 102 is in fluid communication with an inlet of reactive distillation column 103 such that side reactor effluent stream 110 can flow from side reactor 102 to reactive distillation column 103, as shown in FIG. 1.

In embodiments of the disclosure, system 10 includes heater 114 for heating side stream 107 before it enters side reactor 101 and/or cooler 115 for cooling side reactor effluent stream 108 before it enters reactive distillation column 103. Similarly, in embodiments of the disclosure, system 10 includes heater 116 for heating side stream 109 before it enters side reactor 102 and/or cooler 117 for cooling side reactor effluent stream 110 before it enters reactive distillation column 103.

With the configuration of system 10, as shown in FIG. 1, with two side reactors, namely side reactor 101 and side reactor 102, system 10 can operate with side reactor 101 or side reactor 102 while the other side reactor is out of service, for example to regenerate catalyst in the side reactor that is out of service. In this way, continuous operation of system 10 or less down time of system 10, due to regeneration of side reactor catalyst, can be achieved. It should be noted that, in embodiments of the disclosure, side reactor 101 and side reactor 102 can be connected to reactive distillation column 103 at the same locations for inflow and outflow such that side reactor 101 and side reactor 102, can each be a substitute for the other, with respect to withdrawal and processing of a side stream at particular location of reactive distillation column 103. As shown in FIG. 1, in embodiments of the disclosure, side reactor 101 and side reactor 102 can be connected to different locations of reactive distillation column 103 and side reactor 101 and side reactor 102 can be operated concurrently or separately with reactive distillation column 103.

According to embodiments of the disclosure, system 10 further includes pre-distillation reactor 100. Pre-distillation reactor 100, in embodiments of the disclosure, is configured to react feed stream 105, which is fed to pre-distillation reactor 100 by unit 104 (e.g., a pump) to produce distillation column feed stream 106, as shown in FIG. 1. Pre-distillation reactor 100 may include therein a pre-distillation reactor catalyst (e.g., Re/γ-Alumina and/or K/γ-Alumina) for catalyzing a metathesis reaction. According to embodiments of the disclosure, in system 10, the same type of metathesis reaction occurs in pre-distillation reactor 100, reactive distillation column 103, side reactor 101, and/or side reactor 102. An inlet of pre-distillation reactor 100 is in fluid communication with an outlet of unit 104 such that feed stream 105 can flow to pre-distillation reactor 100 and an outlet of pre-distillation reactor 100 is in fluid communication with an inlet of reactive distillation column 103 such that distillation column feed 106 can flow from pre-distillation reactor 100 to reactive distillation column 103, as shown in FIG. 1.

According to embodiments of the disclosure, in system 10, a metathesis reaction is carried out in several stages including different stages of reactive distillation column 103 as well as stages provided by side reactor 101 and/or side reactor 102. Similarly, in embodiments of the disclosure, in system 10, a metathesis reaction is carried out in several stages including different stages of reactive distillation column 103, stages provided by side reactor 101 and/or side reactor 102, and/or a stage provided by pre-distillation reactor 100. It should be noted that although FIG. 1 shows system 10 with two side reactors, system 10, in embodiments of the disclosure, could be operated with more side reactors; for example, one to six side reactors can be used at different locations (stages) of reactive distillation column 103 starting from reboiler 119 up to the condenser 118. Since the catalysts of side reactor 101 and side reactor 102 are located outside of the reactive distillation column 103, such catalysts can be regenerated easily if deactivated, especially in embodiments where two side reactors are present at every location, to ensure that one is in service while the other is being regenerated. A further benefit of system 10, where a plurality of side reactors are involved, according to embodiments of the disclosure, is that it is extremely useful when reaction conditions (temperature and pressure) are out of synchronization with distillation conditions.

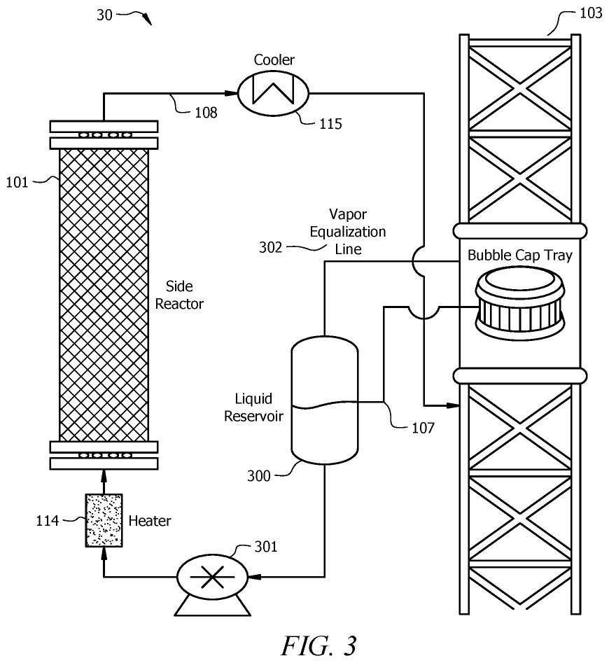

FIG. 3 shows system 30 for producing a product by metathesis reaction according to embodiments of the disclosure. FIG. 3 shows process flow diagram for accessories that can be utilized to connect a side reactor, such as side reactor 101 to reactive distillation column 103, according to embodiments of the disclosure. FIG. 3 shows system 30 includes liquid reservoir holding pot 300 (which can be provided with a level indicator), pump 301 for side stream 107 (liquid), heater 114, cooler 115 for side reactor effluent stream 108, and pressure equalization line 302. In embodiments of the disclosure, all the liquid stream (side stream 107) can be passed through side reactor 101 or only a part of the liquid stream (side stream 107) can be passed through side reactor 101.

### Methods of Producing a Product by Metathesis Reaction

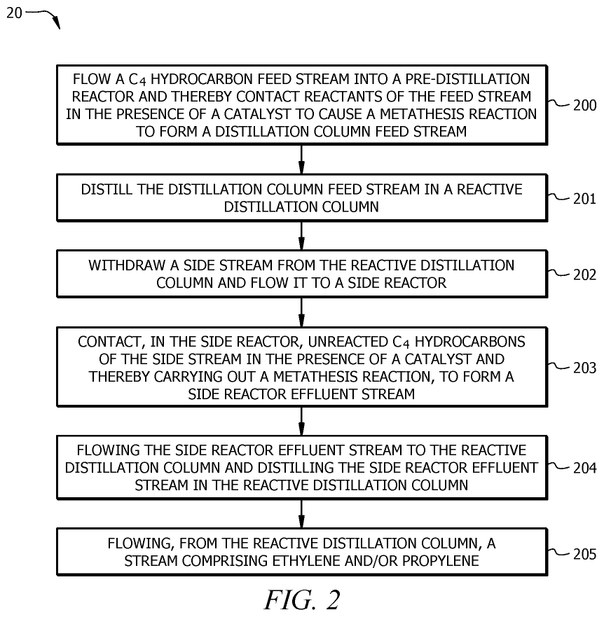

FIG. 2 shows method 20 for producing a product by metathesis reaction, according to embodiments of the disclosure. Method 20 can be implemented by system 10 in embodiments of the disclosure.

Method 20, in embodiments of the disclosure includes, at block 200, flowing feed stream 105 into pre-distillation reactor 100 and thereby contact reactants of feed stream 105 in the presence of a catalyst disposed in pre-distillation reactor 100. According to embodiments of the disclosure, feed stream 105 comprises C₄ hydrocarbons and the catalyst within pre-distillation reactor 100 comprises Re/γ-Alumina and/or K/γ-Alumina. According to embodiments of the disclosure, pre-distillation reactor carries out and catalyzes the metathesis of C₄ hydrocarbons to form distillation column feed stream 106, which comprises ethylene and/or propylene and unreacted C₄ hydrocarbons. According to embodiments of the disclosure, the metathesis reaction of C₄ hydrocarbons comprises isomerization of 1-butene to trans-2-butene and cis-2-butene and isomerization of trans-2-butene and cis-2-butene to 1-butene. According to embodiments of the disclosure, feed stream 105 comprises 0 to 70 mass % 1-butene, 0 to 30 mass % n-butane, 0 to 30 mass % i-butane, 0 to 50 mass % trans-2-butene, and 0 to 30 mass % cis-2-butene. Distillation column feed stream 106, in embodiments of the disclosure, comprises 1-butene, n-butane, mass % i-butane, mass % trans-2-butene, and mass % cis-2-butene.

According to embodiments of the disclosure, method 20 further includes flowing distillation column feed stream 106 to reactive distillation column 103 and distilling distillation column feed stream 106 within reactive distillation column 103, at block 201. At block 202, method 20 involves, in embodiments of the disclosure, withdrawing side stream 107 from reactive distillation column 103 and flowing it to side reactor 101. According to embodiments of the disclosure, side stream 107 can be heated or cooled before it is flowed to side reactor 101. For example, as shown in FIG. 1, in embodiments of the disclosure, side stream 107 is heated with heater 114 before flowing side stream 107 to side reactor 101. According to embodiments of the disclosure, block 203 involves contacting, in side reactor 101, unreacted C₄ hydrocarbons of side stream 107 in the presence of a catalyst and thereby carrying out a metathesis reaction, to form side reactor effluent stream 108. In embodiments of the disclosure, the catalyst inside side reactor 101 comprises Re/γ-Alumina and/or K/γ-Alumina. According to embodiments of the disclosure, the reaction conditions, at block 203, for the metathesis reaction in side reactor 101, comprise a temperature of 40 to 450° C., a pressure of 2.0 to 40 bar g, and a WHSV of 0.2 to 10 l/hr either in gas or liquid phase. According to embodiments of the disclosure, the metathesis reaction of C₄ hydrocarbons at block 203 in side reactor 101 comprises isomerization of 1-butene to trans-2-butene and cis-2-butene and isomerization of trans-2-butene and cis-2-butene to 1-butene. According to embodiments of the disclosure, side stream 107 comprises 1-butene, n-butane, i-butane, trans-2-butene, and cis-2-butene. Side reactor effluent stream 108, in embodiments of the disclosure, comprises 1-butene, n-butane, i-butane, trans-2-butene, and cis-2-butene.

In embodiments of the disclosure, the temperature of operation of the side reactors, such as side reactor 101, differs from the operating temperature of reactive distillation column 103 by 40 to 450° C. with appropriate heat integrated heaters and coolers such as heater 114 and cooler 115. In embodiments of the disclosure, the pressure of operation of the side reactors, such as side reactor 101, differs from the column operating pressure by 2 to 40 bars. Side reactor 101 can be operated either in gas or liquid phase, according to embodiments of the disclosure. In embodiments of the disclosure, side reactor 102 can be operated similarly as described above with respect to side reactor 101 and side reactor 102 and side reactor 101 can be operated concurrently or separately with reactive distillation column 103.

At block 204, method 20 includes flowing side reactor effluent stream 108 to reactive distillation column 103 and distilling side reactor effluent stream 108 within reactive distillation column 103. According to embodiments of the disclosure, side reactor effluent stream 108 can be heated or cooled before it is flowed to reactive distillation column 103. For example, in embodiments of the disclosure, as shown in FIG. 1, side reactor effluent stream 108 can be cooled with cooler 115, before flowing side reactor effluent stream 108 to reactive distillation column 103. In embodiments of the disclosure, the catalyst inside reactive distillation column 103 comprises Re/γ-Alumina and/or K/γ-Alumina. In reactive distillation column 103, according to embodiments of the disclosure, metathesis reaction of C₄ hydrocarbons is carried out and concurrently the products formed by that metathesis reaction is separated from the C₄ hydrocarbon reactants. According to embodiments of the disclosure, the metathesis reaction at block 204 comprises isomerization of 1-butene to trans-2-butene and cis-2-butene and isomerization of trans-2-butene and cis-2-butene to 1-butene.

According to embodiments of the disclosure, block 205 involves flowing, from reactive distillation column 103, top vapor stream 111 comprising, top liquid stream 112, and bottoms stream 113.

Although embodiments of the present disclosure have been described with reference to blocks of FIG. 2 it should be appreciated that operation of the present disclosure is not limited to the particular blocks and/or the particular order of the blocks illustrated in FIG. 3. Accordingly, embodiments of the disclosure may provide functionality as described herein using various blocks in a sequence different than that of FIG. 2.

The systems and processes described herein can also include various equipment that is not shown and is known to one of skill in the art of chemical processing. For example, some controllers, piping, computers, valves, pumps, heaters, thermocouples, pressure indicators, mixers, heat exchangers, and the like may not be shown.

As part of the disclosure of the present disclosure, specific examples are included below. The examples are for illustrative purposes only and are not intended to limit the disclosure. Those of ordinary skill in the art will readily recognize parameters that can be changed or modified to yield essentially the same results.

### EXAMPLE

### (Metathesis Reaction on a C₄ Feed)

Experiments were conducted that involved carrying out a metathesis reaction on a C₄ feed that comprises 1-butene trans/cis-2-butene along with inert C₄ components like n-butane, etc.

Two different feed compositions were evaluated, namely (1) Feed A: Low 1-butene concentration (about 15 mol. %) which typically could be obtained from the ethane steam cracker and (2) Feed B: High 1-butene concentration (about 60 mol. %) which typically could be obtained from a refinery.

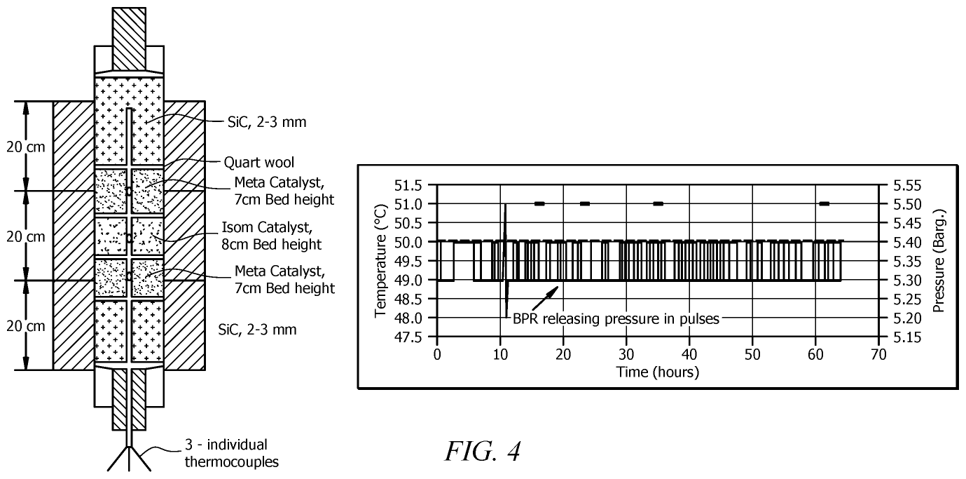

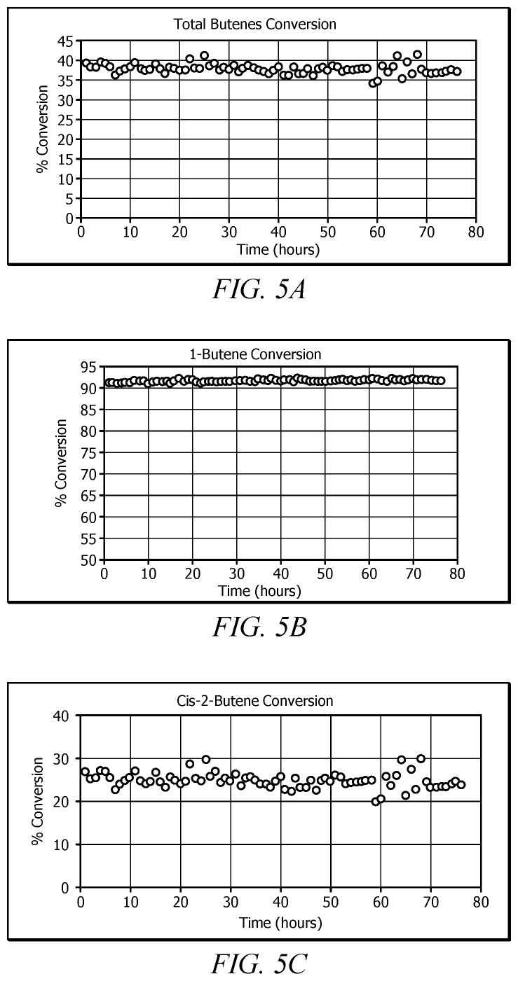

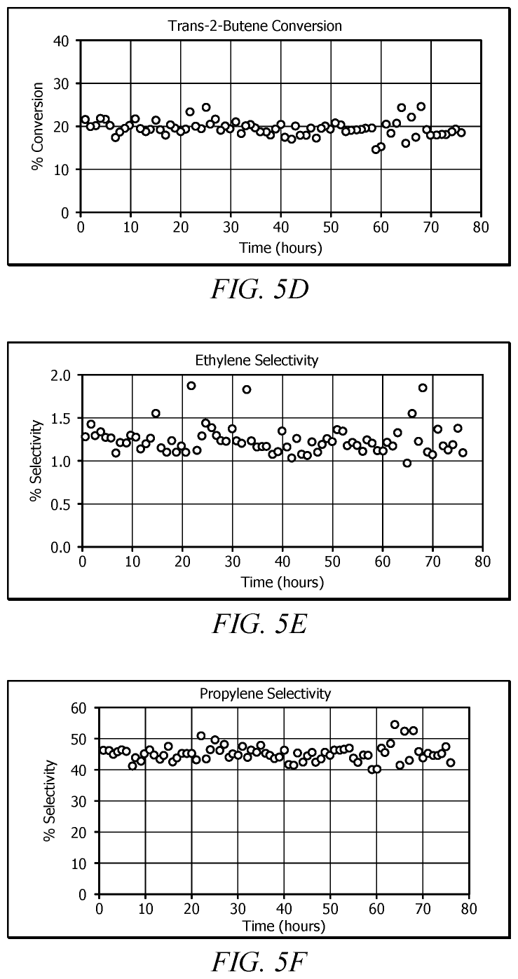

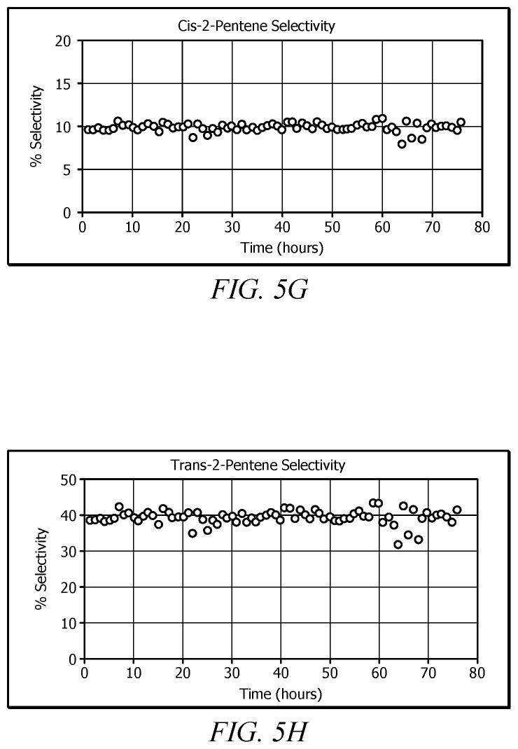

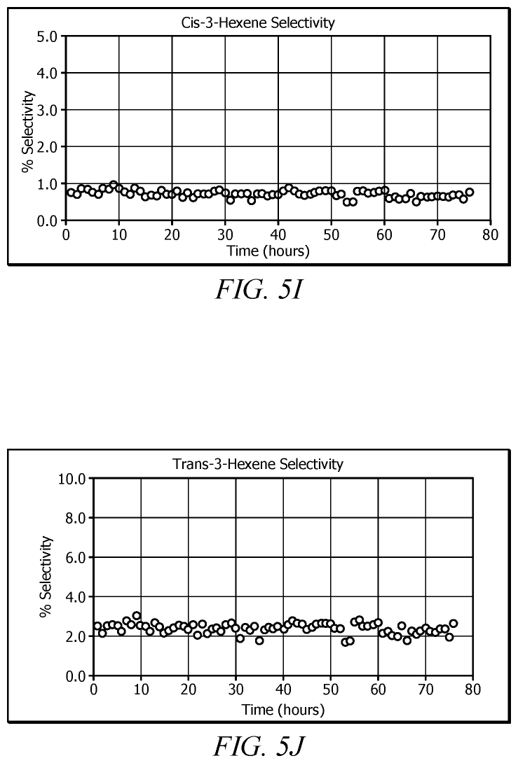

Laboratory experiments were conducted using a packed plug flow reactor at 50° C. with C₄ feed for metathesis and isomerization reaction. FIG. 4 shows a catalyst loading diagram for experiments for the metathesis reaction on a C₄ feed and a plot of temperature and pressure conditions versus time. Reaction conditions are shown below. Results are shown in FIGS. 5A to 5J.

Scaled-up Catalyst - B1 and Reaction Conditions

| Meta Catalyst | 5.4 wt. % re (commercial) Alumina (Sasol HD) | |
|---|---|---|
| Isom Catalyst | 8 wt. % K/Alumina (Sasol HD) | |
| Catalyst Stack | Meta/Isom/Meta | |
| Configuration | | |
| Reaction Conditions: | | |
| Temperature | 50° | C. |
| Pressure | 5.4 | Barg |

| | |
|---|---|
| WHSV | 0.43/hr |
| Feed Composition* | 1-B 26%: 2-B 74% |

*Feed composition with internal standard: 1-butene 23.9%, n-butane 3.8%, i-butane 1.3%, trans-2-butene 49.8%, cis-2-butene 21.2%.

A reactor model using Aspen Plus was developed to correlate the experimental results to the predicted Aspen Plus reactor model. The reaction network used in Aspen Plus is shown below in Table 1.

TABLE 1

| | | Reaction | | | |
|---|---|---|---|---|---|
| No. | Name | Class | Active | Reversible | Stoichiometry |
| 94 | L94 | POWERLAW | [symbol] | [symbol] | 1-butene + T2-butene → propylene(mixed) + |
| | | | | | T2-pente(mixed) |
| 95 | L95 | POWERLAW | [symbol] | [symbol] | 1-butene + C2-butene → propylene(mixed) + |
| | | | | | C2-pentene(mixed) |
| 102 | L102 | POWERLAW | [symbol] | [symbol] | 2 1-butene → ethylene(mixed) + |
| | | | | | T3-hexen(mixed) |
| 103 | L103 | POWERLAW | [symbol] | [symbol] | 2 1-butene → ethylene(mixed) + |
| | | | | | C3-hexene(mixed) |
| 104 | L104 | POWERLAW | [symbol] | [symbol] | 1-butene + propylene → ethylene(mixed) + |
| | | | | | T2-pentene(mixed) |
| 105 | L105 | POWERLAW | [symbol] | [symbol] | 1-butene + propylene → ethylene(mixed) + |
| | | | | | C2-pente(mixed) |
| 111 | L111 | POWERLAW | [symbol] | [symbol] | T2-butene → 1-butene(mixed) |
| 112 | L112 | POWERLAW | [symbol] | [symbol] | C2-butene → 1-butene(mixed) |
| 61 | L61 | POWERLAW | [symbol] | [symbol] | 1-butene + T2-pentene → propylene(mixed) + |
| | | | | | T3-hexene(mixed) |
| 62 | L62 | POWERLAW | [symbol] | [symbol] | 1-butene + C2-pentene → propylene(mixed) + |
| | | | | | C3-hexene(mixed) |

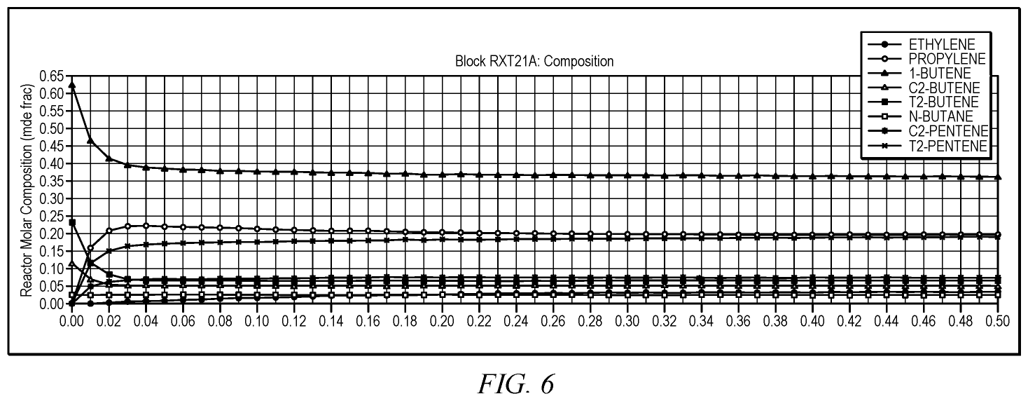

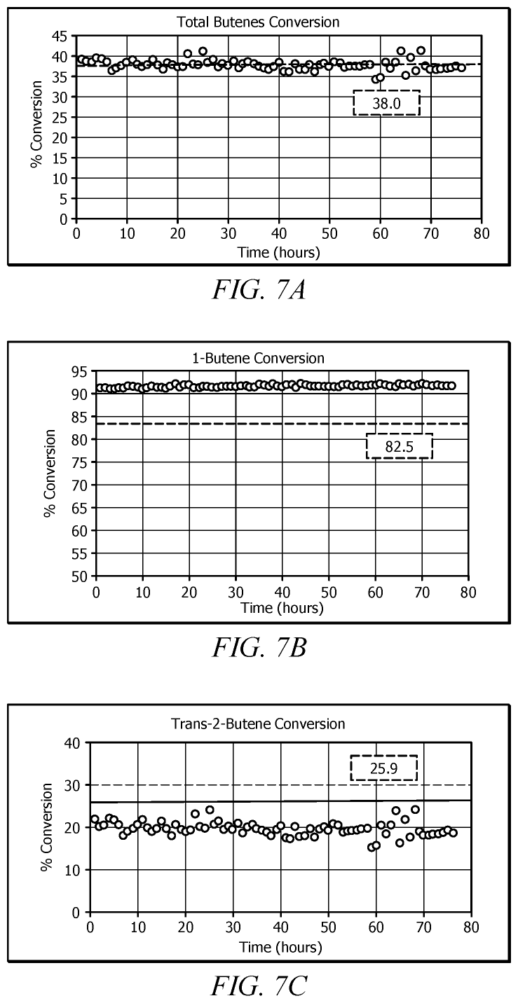

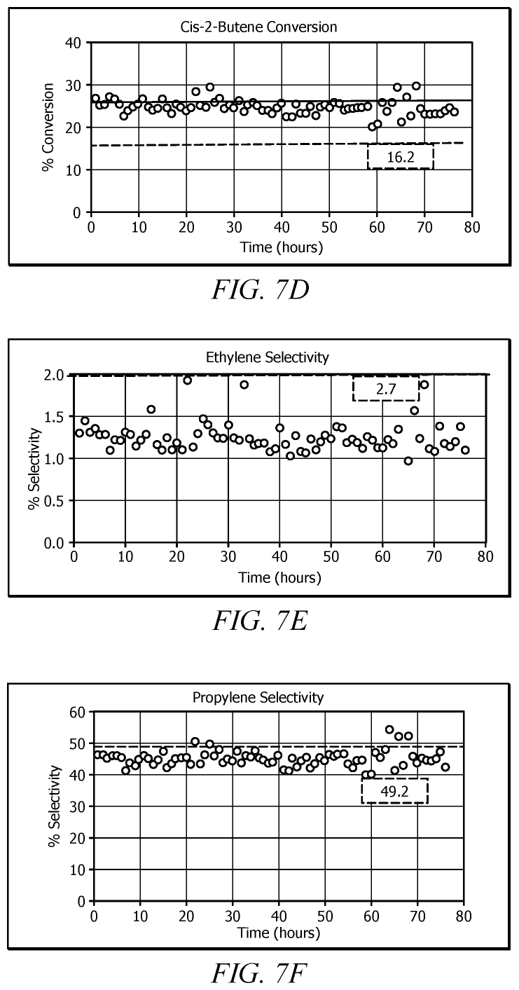

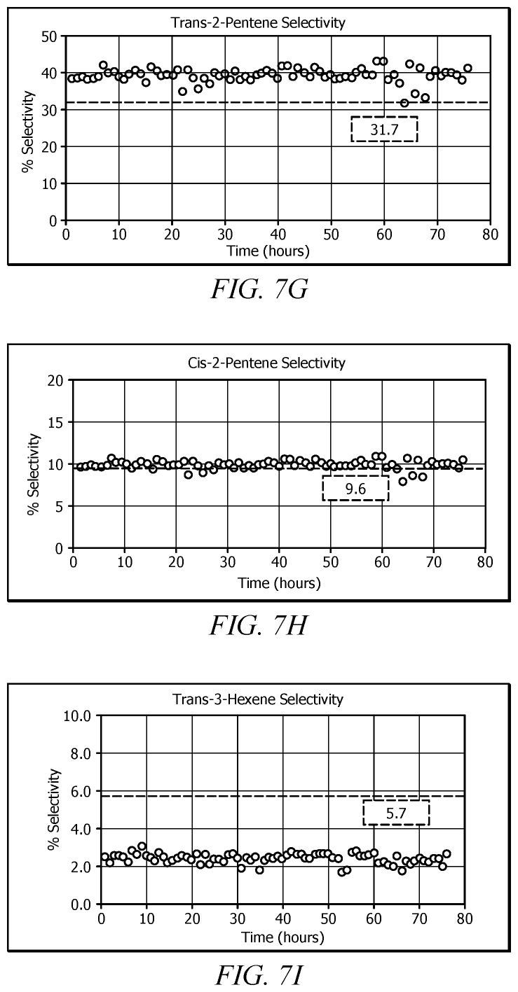

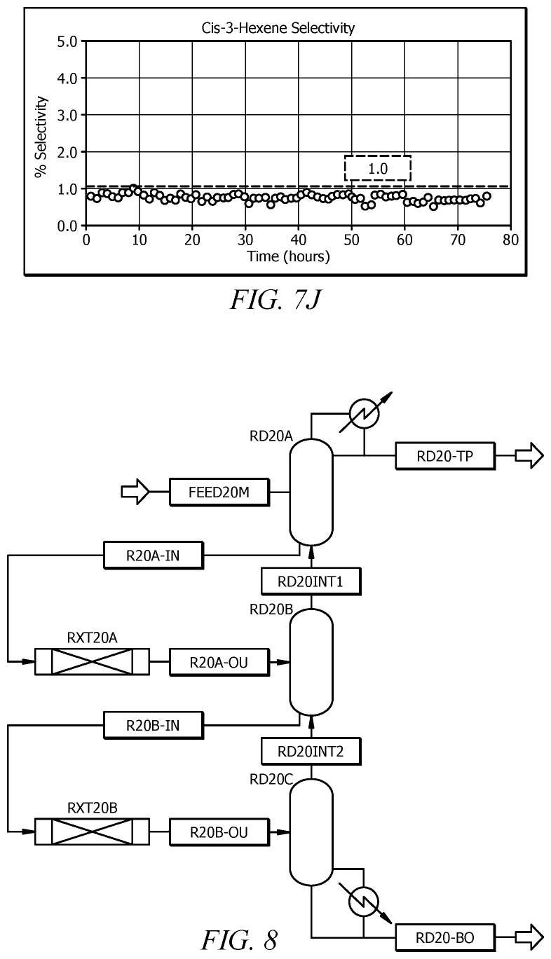

A predicted plug flow reactor profile is shown in FIG. 6. FIG. 7A to 7J show the experimental outlet reactor composition vs the Aspen predicted values.

Aspen Plus simulations were performed to evaluate the performance of commercial plant process using a standalone plug flow reactor and a reactive pump-around process. A feed flow of 18.024 tons/h was used for all the simulations. Two different feed compositions feed-A (containing low 1-butene) and feed-B (containing high 1-butene) were evaluated. The setup of the Aspen Plus program is shown in FIG. 8. The column was broken into three sections (RD20A, RD20B, and RD20C) to aid in convergence of the program. The full liquid from the column was passed into the two side-reactors (RXT20A and RXT20B). The side-reactors were operated at the inlet liquid temperature from the column and liquid was returned back to the column at the outlet temperature of the side reactor. Vapor-liquid flashing was allowed in the side reactors. In FIG. 8, FEED20M is the feed to the column, R20A-IN is the side stream flowing into RXT20A, R20A-OU is the reactor effluent flowing to the column, R20B-IN is the side stream flowing into RXT20B, R20B-OU is the reactor effluent flowing to the column, RD20INT2 is intermediate flow between RD20C and RD20B, RD20INT1 is intermediate flow between RD20B and RD20A, RD20-TP is distillate, and RD20-BO is bottoms.

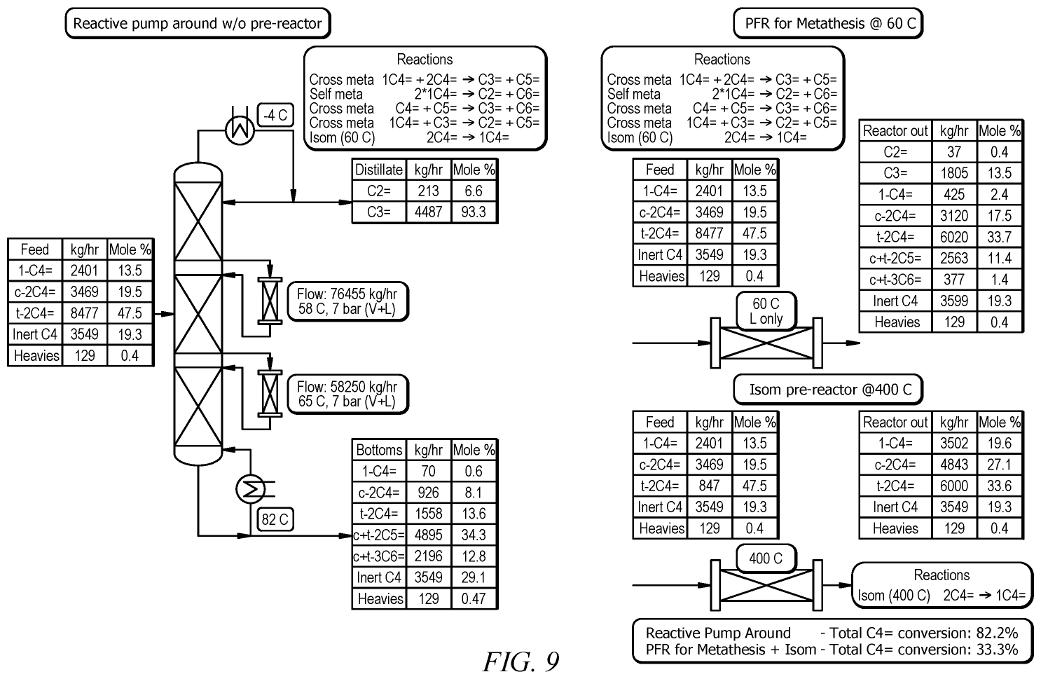

FIG. 9 shows the Aspen Plus simulations for both a stand-alone plug flow reactor and a reactive pump-around process using two external side reactors when feed-A was used. As can be seen from this figure, the total conversion of reactive C₄ olefins increased from 33.3% in the case when a stand-alone plug flow reactor was used and increased to 82.2% when a reactive pump-around process was used. FIG. 9 shows the composition of the outlet streams and the compositions in mol. %. Side reactor flow rates are also shown in FIG. 9.

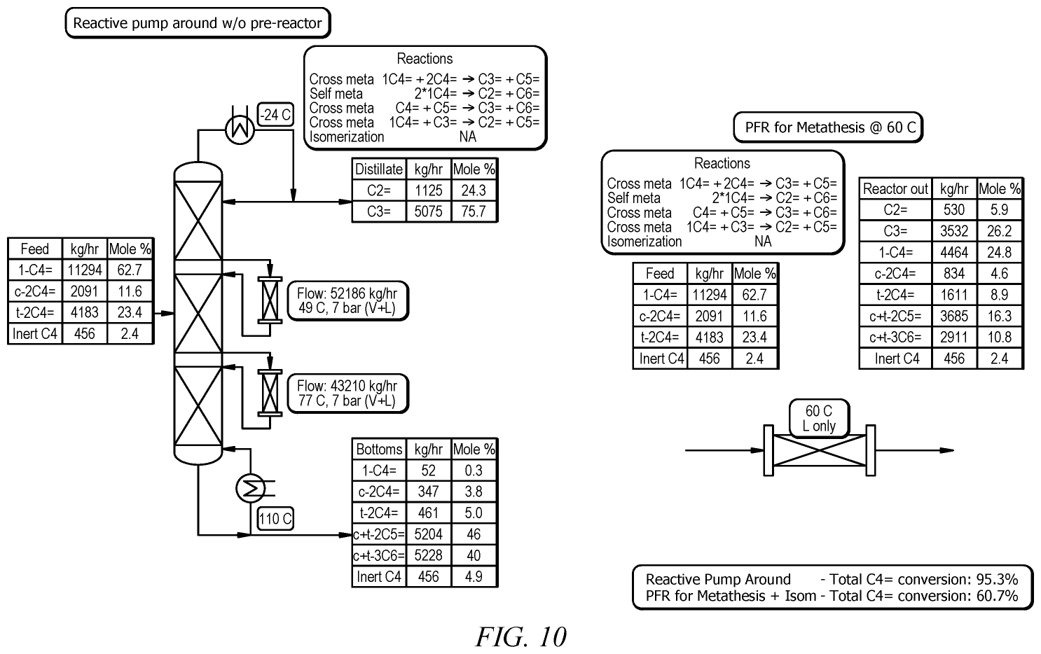

FIG. 10 shows the Aspen Plus simulations for both a stand-alone plug flow reactor and a reactive pump-around process using two external side reactors when feed-A was used. As can be seen from this figure the total conversion of reactive C₄ olefins increased from 60.7% in the case when a stand-alone plug flow reactor was used and increased to 95.3% when a reactive pump-around process was used. FIG. 10 shows the composition of the outlet streams and the compositions in mole %. Side reactor flow rates are also shown in FIG. 10.

It should be noted that embodiments of the disclosure as described with respect to method 20 can be implemented such that the values for corresponding streams and conditions shown in FIG. 9 are in a range of less than 10% of the value shown in FIG. 9 to more than 10% of the value shown in FIG. 9. It should be noted that embodiments of the disclosure as described with respect to method 20 can be implemented such that the values for corresponding streams and conditions shown in FIG. 10 are in a range of less than 10% of the value shown in FIG. 10 to more than 10% of the value shown in FIG. 10.

In the context of the present invention, at least the following 20 embodiments are disclosed. Embodiment 1 is a method of producing a product by metathesis reaction. The method includes distilling a distillation column feed stream in a distillation column and withdrawing a side stream from the distillation column. The method further includes reacting, in a first side reactor, reactants of the side stream, by metathesis reaction, to form a first side reactor effluent stream. The method still further includes distilling the first side reactor effluent stream in the distillation column, and flowing, from the distillation column, a stream containing product. Embodiment 2 is the method of embodiment 1, wherein the metathesis reaction is an equilibrium limited chemical reaction. Embodiment 3 is the method of any of embodiments 1 or 2, wherein the metathesis reaction is one or more of the following: reacting C₂ to C₁₂ olefins, isomerization reactions of C₆ compounds, etherification reactions of C₄ and C₅ olefins with one or more of the following alcohols: methanol, ethanol, and isoamylalcohol. Embodiment 4 is the method of any of embodiments 1 to 3, wherein the distillation column feed stream contains C₄ hydrocarbons. Embodiment 5 is the method of any of embodiments 1 to 4, wherein the metathesis reaction includes isomerization of 1-butene to trans-2-butene and/or cis-2-butene, isomerization of trans-2-butene to 1-butene, or isomerization of cis-2-butene to 1-butene. Embodiment 6 is the method of any of embodiments 1 to 5, wherein reaction conditions for the metathesis reaction include a temperature of 40 to 450° C., a pressure of 2.0 to 40 bar g, and a WHSV of 0.2 to 10 l/hr either in gas and/or liquid phase. Embodiment 7 is the method of any of embodiments 1 to 6, wherein the metathesis reaction is catalyzed by a side reactor catalyst that contains Re/γ-Alumina and/or K/γ-Alumina. Embodiment 8 is the method of any of embodiments 1 to 7, wherein the product is ethylene and/or propylene. Embodiment 9 is the method of any of embodiments 1 to 8, wherein the distillation column includes a packed configuration and/or a tray configuration. Embodiment 10 is the method of any of embodiments 1 to 9, further including withdrawing a plurality of side streams from the distillation column and flowing each of the plurality of side streams to a different one of a plurality of side reactors, and reacting reactants of the plurality of side streams, by metathesis reaction, in the plurality of side reactors, to produce a plurality of side reactor effluent streams, each of the plurality of side reactor effluent streams flowing from a different one of each of the plurality of side reactors. The method further includes distilling each of the plurality of effluent streams in the distillation column. Embodiment 11 is the method of any of embodiments 1 to 10, wherein one or more of the side reactors include a packed bed continuous plug flow reactor and/or a CSTR. Embodiment 12 is the method of any of embodiments 1 to 11, wherein temperature of operation of one or more of the plurality of side reactors differ from the distillation column operating temperature by 40 to 450° C. Embodiment 13 is the method of any of embodiments 1 to 12, wherein pressure of operation of one or more of the plurality of side reactors differ from the distillation column operating pressure by 2 to 40 bars. Embodiment 14 is the method of any of embodiments 1 to 13, wherein one or more of the plurality of side reactors are operated such that the reactants are in gas or liquid phase or a combination thereof. Embodiment 15 is the method of any of embodiments 1 to 14, further including contacting, in a pre-distillation reactor, reactants of a feed stream in presence of a pre-distillation reactor catalyst and thereby carry out a metathesis reaction that forms the distillation column feed stream. Embodiment 16 is the method of any of embodiments 1 to 15, wherein the feed stream contains C₄ hydrocarbons. Embodiment 17 is the method of any of embodiments 1 to 16, wherein the feed stream contains 0 to 70 mass % 1-butene, 0 to 30 mass % n-butane, 0 to 30 mass % i-butane, 0 to 50 mass % trans-2-butene, and 0 to 30 mass % cis-2-butene. Embodiment 18 is the method of any of embodiments 1 to 17, wherein the pre-distillation reactor catalyst contains Re/γ-Alumina and/or K/γ-Alumina. Embodiment 19 is the method of any of embodiments 1 to 18, wherein the distillation column feed stream contains ethylene and/or propylene and unreacted C₄ hydrocarbons.

Embodiment 20 is a method of producing ethylene and/or propylene by metathesis reaction of C₄ hydrocarbons. The method includes contacting, in a pre-distillation reactor, reactants of a feed stream containing C₄ hydrocarbons in the presence of a first catalyst containing Re/γ-Alumina and/or K/γ-Alumina and thereby carrying out a metathesis reaction that forms a distillation column feed stream containing ethylene and/or propylene and unreacted C₄ hydrocarbons. The method further includes distilling the distillation column feed stream in a distillation column, and withdrawing a side stream from the distillation column. The method still further includes contacting, in a first side reactor, unreacted C₄ hydrocarbons of the side stream in the presence of a second catalyst containing Re/γ-Alumina and thereby carrying out a metathesis reaction, to form a first side reactor effluent stream. The method also includes distilling the first side reactor effluent stream in the distillation column, flowing, from the distillation column, a stream containing ethylene and/or propylene.

All embodiments described above and herein can be combined in any manner unless expressly excluded.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. A method of producing a product by metathesis reaction, the method comprising:
distilling a distillation column feed stream in a distillation column;
withdrawing a side stream from the distillation column;
reacting, in a first side reactor, reactants of the side stream, by metathesis reaction, to form a first side reactor effluent stream;
distilling the first side reactor effluent stream in the distillation column; and
flowing, from the distillation column, a stream comprising product.

2. The method of claim 1, wherein the metathesis reaction is an equilibrium limited chemical reaction and/or wherein the metathesis reaction is one or more of the following: reacting C₂ to C₁₂ olefins, isomerization reactions of C₆ compounds, etherification reactions of C₄ and C₅ olefins with one or more of the following alcohols: methanol, ethanol, and isoamylalcohol.

3. The method of claim 1, wherein the distillation column feed stream comprises C₄ hydrocarbons.

4. The method of claim 1, wherein the metathesis reaction comprises isomerization of 1-butene to trans-2-butene and/or cis-2-butene, isomerization of trans-2-butene to 1-butene, or isomerization of cis-2-butene to 1-butene.

5. The method of claim 1, wherein reaction conditions for the metathesis reaction comprise a temperature of 40 to 450° C., a pressure of 2.0 to 40 bar g, and a WHSV of 0.2 to 10 l/hr either in gas and/or liquid phase.

6. The method of claim 1, wherein the metathesis reaction is catalyzed by a side reactor catalyst that comprises Re/γ-Alumina and/or K/γ-Alumina.

7. The method of claim 1, wherein the product is ethylene and/or propylene.

8. The method of claim 1, wherein the distillation column comprises a packed configuration and/or a tray configuration.

9. The method of claim 1, further comprising withdrawing a plurality of side streams from the distillation column and flowing each of the plurality of side streams to a different one of a plurality of side reactors, and reacting reactants of the plurality of side streams, by metathesis reaction, in the plurality of side reactors, to produce a plurality of side reactor effluent streams, each of the plurality of side reactor effluent streams flowing from a different one of each of the plurality of side reactors; and
distilling each of the plurality of effluent streams in the distillation column.

10. The method of claim 9, wherein one or more of the side reactors comprise a packed bed continuous plug flow reactor and/or a CSTR.

11. The method of claim 9, wherein temperature of operation of one or more of the plurality of side reactors differ from the distillation column operating temperature by 40 to 450° C.

12. The method of claim 9, wherein pressure of operation of one or more of the plurality of side reactors differ from the distillation column operating pressure by 2 to 40 bars and/or wherein one or more of the plurality of side reactors are operated such that the reactants are in gas or liquid phase or a combination thereof.

13. The method of claim 9, further comprising:
contacting, in a pre-distillation reactor, reactants of a feed stream in presence of a pre-distillation reactor catalyst and thereby carry out a metathesis reaction that forms the distillation column feed stream and/or wherein the feed stream comprises C₄ hydrocarbons, preferably the feed stream comprises 0 to 70 mass % 1-butene, 0 to 30 mass % n-butane, 0 to 30 mass % i-butane, 0 to 50 mass % trans-2-butene, and 0 to 30 mass % cis-2-butene.

14. The method of claim 13, wherein the pre-distillation reactor catalyst comprises Re/γ-Alumina and/or K/γ-Alumina and/or wherein the distillation column feed stream comprises ethylene and/or propylene and unreacted C₄ hydrocarbons.

15. A method of producing ethylene and/or propylene by metathesis reaction of C₄ hydrocarbons, the method comprising:
contacting, in a pre-distillation reactor, reactants of a feed stream comprising C₄ hydrocarbons in the presence of a first catalyst comprising Re/γ-Alumina and/or K/γ-Alumina and thereby carrying out a metathesis reaction that forms a distillation column feed stream comprising ethylene and/or propylene and unreacted C₄ hydrocarbons;
distilling the distillation column feed stream in a distillation column;
withdrawing a side stream from the distillation column;
contacting, in a first side reactor, unreacted C₄ hydrocarbons of the side stream in the presence of a second catalyst comprising Re/γ-Alumina and thereby carrying out a metathesis reaction, to form a first side reactor effluent stream;
distilling the first side reactor effluent stream in the distillation column; and
flowing, from the distillation column, a stream comprising ethylene and/or propylene.

16. The method of claim 2, further comprising withdrawing a plurality of side streams from the distillation column and flowing each of the plurality of side streams to a different one of a plurality of side reactors, and reacting reactants of the plurality of side streams, by metathesis reaction, in the plurality of side reactors, to produce a plurality of side reactor effluent streams, each of the plurality of side reactor effluent streams flowing from a different one of each of the plurality of side reactors; and
distilling each of the plurality of effluent streams in the distillation column.

17. The method of claim 3, further comprising withdrawing a plurality of side streams from the distillation column and flowing each of the plurality of side streams to a different one of a plurality of side reactors, and reacting reactants of the plurality of side streams, by metathesis reaction, in the plurality of side reactors, to produce a plurality of side reactor effluent streams, each of the plurality of side reactor effluent streams flowing from a different one of each of the plurality of side reactors; and
distilling each of the plurality of effluent streams in the distillation column.

18. The method of claim 4, further comprising withdrawing a plurality of side streams from the distillation column and flowing each of the plurality of side streams to a different one of a plurality of side reactors, and reacting reactants of the plurality of side streams, by metathesis reaction, in the plurality of side reactors, to produce a plurality of side reactor effluent streams, each of the plurality of side reactor effluent streams flowing from a different one of each of the plurality of side reactors; and
distilling each of the plurality of effluent streams in the distillation column.

19. The method of claim 5, further comprising withdrawing a plurality of side streams from the distillation column and flowing each of the plurality of side streams to a different one of a plurality of side reactors, and reacting reactants of the plurality of side streams, by metathesis reaction, in the plurality of side reactors, to produce a plurality of side reactor effluent streams, each of the plurality of side reactor effluent streams flowing from a different one of each of the plurality of side reactors; and
distilling each of the plurality of effluent streams in the distillation column.

20. The method of claim 6, further comprising withdrawing a plurality of side streams from the distillation column and flowing each of the plurality of side streams to a different one of a plurality of side reactors, and reacting reactants of the plurality of side streams, by metathesis reaction, in the plurality of side reactors, to produce a plurality of side reactor effluent streams, each of the plurality of side reactor effluent streams flowing from a different one of each of the plurality of side reactors; and
distilling each of the plurality of effluent streams in the distillation column.
